(19) United States
(12) Plant Patent Application Publication
(10) Pub. No.: US 2002/0124289 P1
(43) Pub. Date: Sep. 5, 2002

(54) ' Double Spotty '

(76) Inventors: David W. Fleming, Lincoln, NE (US); Gretchen A. Zwetzig, Lincoln, NE (US)

Correspondence Address: David Fleming and Gretchen Zwetzig, Fleming's Flower Fields, Inc., 8101 S. 14th St., Lincoln, NE, 68512, US

(21) Appl. No.: 09/798,389

(22) Filed: Mar. 5, 2001

(51) Int. Cl. A01H 5/00

(52) U.S. Cl. PLT/272

(57) ABSTRACT

A new and distinct cultivar of winter hardy Dianthus plant named ‘Double Spotty’ is the result of a unique hybridization. This new and distinct cultivar is characterized primarily as to novelty by its extreme cold hardiness to Zone 4, its cherry red flowers like Spotty but having multiple rows of petals. This everblooming cultivar has a nice compact form with vigorous growth.

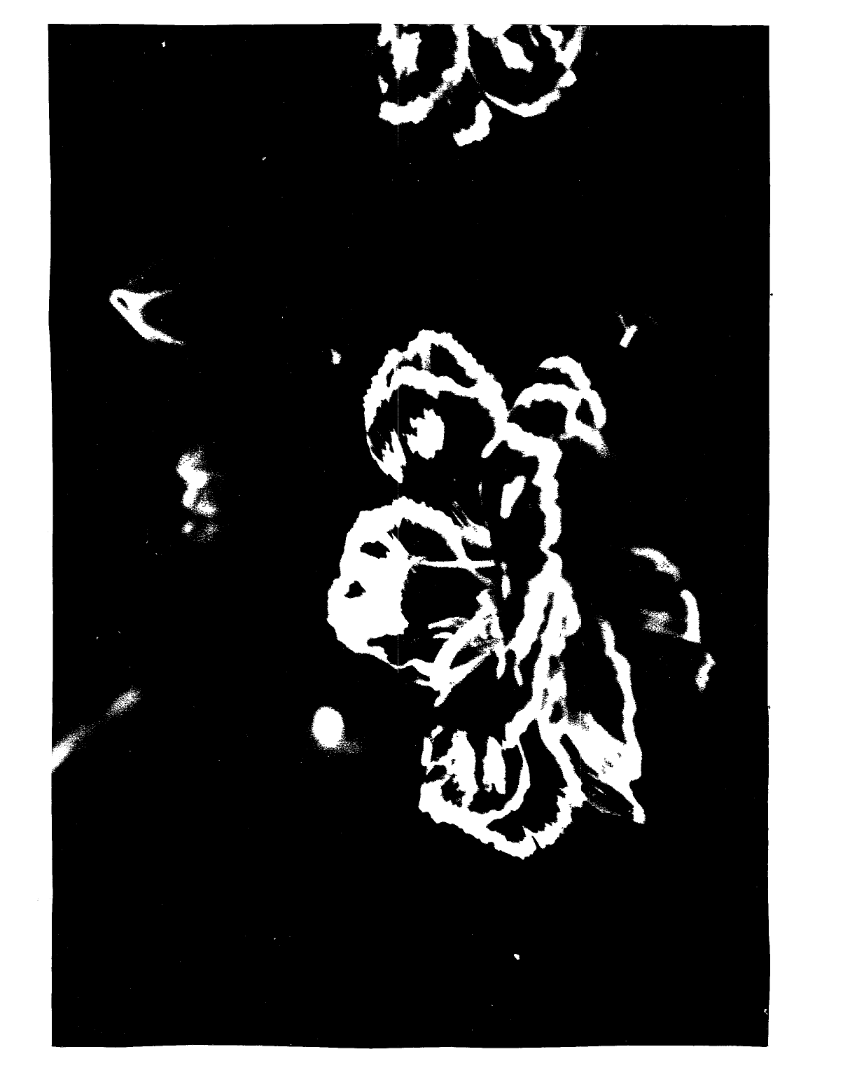

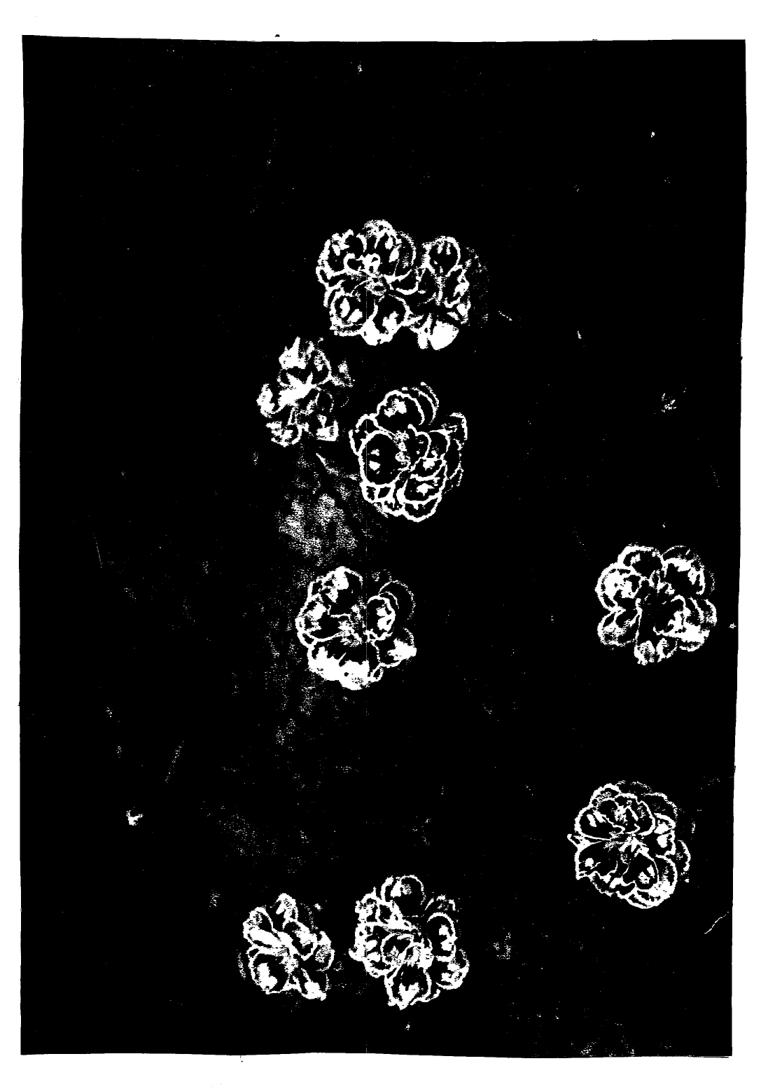

### BACKGROUND OF THE NEW PLANT

The new plant of this invention is the result of a unique hybridization, with the breeding achievement being evidenced in the outstanding combination of characteristics exhibited by this new and distinct Dianthus plant, which include:

(a) Blue-green upright foliage that forms a neat and compact bun and compliments the gardenscape:

(b) Bright red flowers with double spotlike markings on each each petal. The petals are arranged in 2-3 rows, adding to the interest.

(c) The plant being very floriferous with flowers that bloom in the Spring, Summer, and late Summer.

(d) The plant being medium-sized, but vigorous, with compact and uniform growth which gives it unique landscape utility; and

(e) The plant being so hardy that it can consistently withstand winter temperatures of at least −30 degrees Fahrenheit.

### SUMMARY OF THE INVENTION

Double Spotty was the result of a 40—year breeding program. Its ancestry includes D. Grantianapolitanus, D. Plumarius, and D. Neglectus. More specifically, the plant resulted from a selected seedling of Spotty (a Fleming introduction). The seedling which most nearly met all above named standards was selected.

This new plant first bloomed in the summer of 1985 and was selected by David Fleming and Gretchen Zwetzig on David Fleming's property in Lincoln, Nebr. Asexual propagation of the plant by cuttings and root division in Lincoln, Nebr; has shown that the unique and distinguishing features of the plant are faithfully transmitted from generation to generation and appear to be fixed.

Since its origin, the plant has bloomed from midsummer-until frost, while exhibiting the above-mentioned distinctive characteristics. This hardy Dianthus plant contributes to the market with its sheer beauty, its compact growth habit, its great resistance to disease and insects, its stability through extremes in rain and drought, and its extreme hardiness.

### BRIEF DESCRIPTION OF THE DRAWINGS

The flower, bud, foliage, and growth habit of the plant are shown in the attached illustrations. More specifically, sheet 1 shows a close up of the flower and sheet 2 shows the habit of the hybrid. The flower/plant colors are as true as is reasonably possible to attain in photographic illustrations of this type. The colors illustrated may be slightly off due to light reflectance.

### DESCRIPTION OF THE NEW PLANT

What follows is a detailed description of the new cultivar. The specific color designations set forth by PLATE and number designations are in accordance with the Dictionary of Color (Maerz and Paul), while general color recitations are consistent with ordinary American color Terminology.

Double Spotty has not been observed under all possible environmental conditions. It is to be understood that the phenotype may vary significantly with variations in the environment such as temperature, light intensity, and day length, without, however any difference in genotype of the plant. The following botanical characteristics and observations are taken from the plant when grown under normal outdoor conditions in Lincoln, Nebr.

### THE PLANT

Parentage: Seed Parent: Spotty D. plumarius, neglectus, and grantianapolitanus Pollen Parent: Horatio D. neglectus

Origin.—A selected seedling from the stated cross.

Type of plant.—Hardy herbaceous perennial.

Classification.—Hybrid Dianthus plant. (Botanic) Border dianthus (commercial)

Propagation.—Relatively easy to propagate; holds its distinguishing characteristics through successive propagations by cuttings.

Size .—About 7-12″ tall about 12″ wide.

Form.—Symmetrical annual growth from perennial roots.

Habit.—Upright with many uniform branches. Forms a compact bun.

Growth.—Vigorous; medium and compact.

Hardiness.—Hardy to at least −30 degrees Fahrenheit.

General Health.—Plant is very disease resistant; very pest resistant; sturdy through excessive drought or water.

Foliage: 1. Quantity: abundant 2. Shape: Linear Lancealate 3. Size: 5 cm in length. 3 mm width. 4. Color: Silvery Blue-green I-5, PLATE 30, page 83 of the Dictionary of Color. 5. Surface Texture: Glabrous 6. Ribs and Veins: Barely noticeable. 7. Rachis: Barely noticeable.

### THE BUD

Size.—small Diameter: ½ cm Length: 1 ½ cm

Form.—Ovoid

Opening Rate.—Normal

Color of Petals.—When bud is opening, 2-F, PLATE 2, page 27

Sepals: Shape: Jointed at the base with acute character and sharp apexes. The sepals are sturdy to half way up the bud. Color: L-11, Plate 23, page 69.

Calyx: Size: small Shape: Bell-shaped. Splitting: At the base. Overlapping sepals Aspect: Smooth

Flower Stem: Length: To about 15 cm. Character: Erect. Color: A-7, Plate 29, page 81.

### THE FLOWER

Blooming Period.—“Everblooming”. Profuse from Mid-May until early September.

Flower Diameter.—Small to medium.

Borne.—Singly.

Shape.—Almost dome-shaped with several rows of petals (3-5)

Petalage.—Number of petals is 15-20.

Arrangement.—Alternate and overlapping in layers.

Surface Texture.—Soft with satin appearance.

Form.—Fan-shaped and rounded.

Coloration.—Cherry red petals with soft pink markings Petals: 6-K, Plate5, page 33. Markings: 2-F, Plate 22, page 27. Reverse of Petals: 2-F, Plate 2, page 27.

Fragrance.—Sweet and spicy clove.

Reproductive organs: 1. Stamens: Number 3-11. Arrangement: Around the pistils. Anthers: Size- 4 mm. Color: Whitish. Filaments: Length; less than 4 mm. Color: Whitish. 2. Pistils: Number: Two. Styles: Color- White. Stigmas: Color- White. 3. Character of Ovaries: Cone-shaped.

### GENERAL OBSERVATIONS

This plant, as a hybridized hardy everblooming Dianthus, is valuable to the landscape market for its improvements in the addition of rows of petals on the charming spotty dianthus, its all-around vigor, dense and compact breaking action, and adaptation to extreme environments, including the ability to consistently survive winter temperatures of at least −30 degrees Fahrenheit.

1. A new and distinct cultivar of Dianthus plant, as herein shown and described, characterized by its neat and compact blue-green foliage, its enhanced spotty like flowers of cherry red with soft pink spots and its “everblooming” vigor, and its hardiness:
